(19) United States
(12) Patent Application Publication
(10) Pub. No.: US 2011/0294219 A1
(43) Pub. Date: Dec. 1, 2011

(54) USE OF THE FOAMY VIRUS BET PROTEIN FOR INACTIVATING APOBEC

(76) Inventors: Martin Löchelt, Sandhausen (DE); Carsten Münk, Frankfurt (DE)

(21) Appl. No.: 13/180,881

(22) Filed: Jul. 12, 2011

Related U.S. Application Data
(63) Continuation of application No. 11/628,218, filed on Apr. 23, 2007, now abandoned.
(60) Provisional application No. 60/577,342, filed on Jun. 4, 2004.

(51) Int. Cl. C12N 15/85

(52) U.S. Cl. 435/455

(57) ABSTRACT

Described is the foamy virus Bet-mediated inactivation of the mutagenic, genome-modifying and vector-inactivating cellular enzyme ABOBEC. Such inactivation is useful for the treatment or prevention of various diseases, e.g., cancer, or for enhancing the production and genetic stability of gene therapy vectors, preferably retroviral vectors.

This application is a continuation application of U.S. application Ser. No. 11/628,218, filed Apr. 23, 2007; which is a national Stage of International Application PCT/EP2005/006060, filed Jun. 6, 2005; which claims the benefit of U.S. Provisional Application No. 60/577,342, filed Jun. 4, 2004; the above-identified applications are incorporated herein by reference in their entirety.

The present invention relates to the foamy virus Bet-mediated inactivation of the mutagenic, genome-modifying and vector-inactivating cellular enzyme APOBEC. Such inactivation is useful for the treatment or prevention of various diseases, e.g., cancer, or for enhancing the production and genetic stability of gene therapy vectors.

The members of the APOBEC enzyme family (Wiegand et al., EMBO J. May 2004, in press; Argyris et al., Trends Microbiol. 2004, 12(4), pp. 145-148) e.g., AID, APOBEC1, APOBEC3F and APOBEC3G, are cellular cytidine deaminases. Deamination of cytidine is a physiological process which has an important function particularly with respect to inherited and acquired immune responses. AID (activation induced deaminase) seems to modify the immunoglobulin-V-genes (gene conversion and hypermutation, respectively) resulting in antibody diversity. AID attacks upstream of IgC and, thus, induces class switch. APOBEC1 edits the mRNA of apolipoprotein B which is predominantly present in LDL (low density lipoproteins) and VLDL (very low density lipoproteins). Moreover, APOBEC1 is capable of deaminating DNA in vitro. The target of APOBEC3G (CEM15) is retroviral cDNA and the biological role of APOBEC3 is to prevent viral infection by modifying the virally encoded DNA. Moreover, it can be expected that by deamination of tumor suppressor genes (e.g., p53, APC) cancer genesis can be promoted. Finally, at least seven further members of the APOBEC family are known with their biological function in humans being unknown so far.

Members of the APOBEC family might be involved in various pathological processes. The faulty regulation of AID dependent processes is assumed to be responsible for the formation of B cell tumours. In transgenic mice, the faulty expression of AID and APOBEC1 leads to predisposition for cancer genesis. Thus, it can be expected that the inhibition of the biologic activity of APOBEC might have a variety of advantageous effects which can be exploited for therapy. However, at present it is unclear how the inhibition/reduction of the biological activity of APOBEC can be satisfactorily affected.

Therefore, it is the object of the present invention to provide means allowing to inhibit or at least to reduce the biological activity of an APOBEC enzyme.

According to the present invention this is achieved by the subject matters defined in the claims.

It has been found during the experiments leading to the present invention that FV (Foamy Virus) Bet proteins can efficiently inhibit the biological activity of APOBEC. Thus, by functionally inhibiting the cellular APOBEC enzyme by use of FV Bet protein (or the gene encoding it) the occurrence of mutations on cellular or viral genomes, e.g., viral vector genomes, due to deamination of cytidines can be prevented. This approach is useful for gene therapy (using, e.g, retroviral vectors, which can be produced more efficiently and can be stabilized, resulting in an increased biological safety) and for prevention/therapy of diseases which are induced by mutation of particular genes, e.g., mutational inactivation of tumor suppressor genes like p53 in case of tumor genesis.

Moreover, the present invention allows to apply HIV and FV vectors for gene expression in APOBEC positive cells. Foamy viruses (FV; spumavirinae) are a particular group of retroviruses. In some mammalian species, e.g., primates, cats, rodents and cows, they are endemic. In vitro, they exhibit a strong cytopathic effect, however, in vivo, so far the presence of FV in its natural host could not be shown to be associated with any disease. A human FV (HFV) has been characterized, however recent studies indicate that this type of FV is not of human origin but presumably traces back to an infection of a patient with SFV (simian foamy virus). According to recent studies there seems to be no longer any doubt that SFV can be transmitted to humans resulting in a chronic infection without any symptoms of a disease. So far, a secondary transmission from humans to humans could not be detected and this kind of transmission is regarded as being highly unlikely. It is unknown whether genetic changes of the retroviruses after transmission from their natural hosts to humans can influence the course of infection.

Due the benign character of natural FV infections, the broad tropism of FV as well as the further properties of FV which are typical of retroviruses, FV vectors were developed for use in gene therapy. Due to its high activity, the reverse transcriptase of FV is of major interest for scientific/medical studies. Replication of FV is controlled by two promoters, the LTR and a second internal promoter (IP) which is located within the env gene. IP is responsible for the direction of expression of two genes, the transcription factor (Ta) and the accessory protein (Bet). According to earlier publications, Bet was associated with the following functions: Negative regulation of the basal IP activity, maintenance and control of viral persistence, hampering of viral infection.

Accordingly, the present invention relates to a method of preventing the negative effects of an APOBEC enzyme, i.e, undesired deamination of genes, comprising administering to a subject a therapeutically effective amount of an FV Bet protein or the gene encoding said FV Bet protein.

As used herein, the term “FV Bet protein” comprises the natural protein (Löchelt et al., Curr. Tpo. Microbiol. Immunol. 277 (2003), pp. 27-61) as well as proteins exhibiting alterations compared to the natural protein (e.g., substitution, addition and/or deletion of amino acid(s), differing glycosylation pattern) which are still biologically active.

Preferably, for administration, the FV Bet protein is combined with a pharmaceutically acceptable carrier. “Pharmaceutically acceptable” is meant to encompass any carrier, which does not interfere with the effectiveness of the biological activity of the active ingredient and that is not toxic to the host to which it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at an effective dose.

The term “preventing the negative effects of an APOBEC enzyme” as used herein, relates to complete or at least partial inhibition of the deaminating activity of the enzyme.

An “effective amount” refers to an amount of the active ingredient that is sufficient to prevent or at least reduce the negative effects of an APOBEC enzyme. An “effective amount” may be determined using methods known to one skilled in the art (see for example, Fingl et al., The Pharmocological Basis of Therapeutics, Goodman and Gilman, eds. Macmillan Publishing Co., New York, pp. 1-46 ((1975)).

Administration of the suitable compositions may be effected by different ways, e.g. by intravenous, intraperetoneal, subcutaneous, intramuscular, topical or intradermal administration. The route of administration, of course, depends on the kind of therapy and the kind of compound contained in the pharmaceutical composition. The dosage regimen will be determined by the attending physician and other clinical factors. As is well known in the medical arts, dosages for any one patient depends on many factors, including the patient's size, body surface area, age, sex, the particular compound to be administered, time and route of administration, the kind of therapy, general health and other drugs being administered concurrently.

The use of an FV Bet protein according to the present invention also comprises the administration of DNA sequences encoding said compound in such a form that they are expressed in the subject or a desired tissue of the subject.

Recombinant vectors for expression of an FV Bet protein can be constructed according to methods well known to the person skilled in the art; see, e.g., Sambrook, Molecular Cloning A Laboratory Manual, Cold Spring Harbor Laboratory (1989). Preferred recombinant vectors useful for gene therapy are viral vectors, e.g. adenovirus, AAV, herpes virus, vaccinia, or, more preferably, an RNA virus such as a retrovirus. Even more preferably, the retroviral vector is a derivative of a murine or avian retrovirus. Examples of such retroviral vectors which can be used in the present invention are: Moloney murine leukemia virus (MoMuLV), Harvey murine sarcoma virus (HaMuSV), murine mammary tumor virus (MuMTV), Rous sarcoma virus (RSV) and FV. Most preferably, a non-human primate retroviral vector is employed, such as the gibbon ape leukemia virus (GaLV), providing a broader host range compared to murine vectors. Since recombinant retroviruses are defective, assistance is required in order to produce infectious particles. Such assistance can be provided, e.g., by using helper cell lines that contain plasmids encoding all of the structural genes of the retrovirus under the control of regulatory sequences within the LTR. Suitable helper cell lines are well known to those skilled in the art. Said vectors can additionally contain a gene encoding a selectable marker so that the transduced cells can be identified. Moreover, the retroviral vectors can be modified in such a way that they become target specific. This can be achieved, e.g., by inserting a polynucleotide encoding a sugar, a glycolipid, or a protein, preferably an antibody. Those skilled in the art know additional methods for generating target specific vectors. Further suitable vectors and methods for in vitro- or in vivo-gene therapy (including the introduction of the FV Bet encoding nucleotide sequences by lipofection, transfection of naked DNA, RNA-transfer) are described in the literature and are known to the persons skilled in the art; see, e.g., WO 94/29469 or WO 97/00957.

In order to achieve expression only in the target organ, the DNA sequence encoding the FV Bet can also be operably linked to a tissue specific promoter and used for gene therapy. Such promoters are well known to those skilled in the art (see e.g. Zimmermann et al, (1994) Neuron 12, 11-24; Vidal et al., (1990) EMBO J. 9, 833-840; Mayford et al., (1995), Cell 81, 891-904; Pinkert et al., (1987) Genes & Dev. 1, 268-76).

Although the experiments leading to the present invention relate to the inhibition of the biological activity of APOBECG3 it can be expected that the FV Bet protein also shows positive effects as regards the suppression of the activity of further members of the APOBEC family. In a preferred embodiment of the method of the present invention, the member of the APOBEC family belongs to APOBEC3, in particular APOBEC3G and/or APOBEC3F.

The method of the present invention is useful for various purposes with preferred uses being:

(a) Prevention or treatment of diseases associated with the negative effects of an APOBEC protein on genes, i.e., deamination and, thus, mutation/inactivation of a tumor suppressor gene like p53 or APC.
(b) Thus, a particularly preferred use is prevention or treatment of cancer.
(c) Increasing the efficient production of vectors, preferably retroviral vectors, in cells expressing APOBEC, e.g., APOBEC3G and/or APOBEC3F, and/or improving the genetic stability of such vectors.
(d) Studies on the APOBEC-mediated oncogenesis.

Finally, the present invention relates to a method of gene therapy of a disorder associated with (undesired or aberrant) gene deamination comprising introducing into cells of a host subject, an expression vector comprising a nucleotide sequence encoding an FV Bet protein, in operable linkage with a promoter. The present invention is explained by the following examples.

### EXAMPLE 1

### Inactivation of Bet in the Feline and Human Foamy Virus Results in APOBEC-3G-Mediated Genome Mutation

### (A) Experimental System A and Results

Wild-type feline foamy virus (FFV) genomes (pFeFV-7; Zemba et al., Virology 266 (2000), pp. 150-156) containing either a truncated or an intact bet gene (pFeFV-MCS, pFeFV-BBtr: Alke et al., Virology 287 (2001), pp. 310-320) were transfected into feline APOBEC-3G-positive CRFK cells (Dr. Roland Riebe, BFAV, Insel Riems, Germany; Crandell et al., InVitro 1973, 9(3), pp. 176-185). Virions were harvested after 3 days and part of the DNA genomes was amplified by PCR (sense-Primer: 5″-CTTCTGGTTTGGACCTTACC (SEQ ID NO: 5); antisense-Primer: 5″-GTTTTAGTAAGTGTAGCGGCGA (SEQ ID NO: 6)), cloned and sequenced. Sequence analysis showed that only in bet-deleted genomes the APOBEC-3G-specific mutations (C to T on the minus provirus DNA strand) occurred. The amplified genomes from bet-containing FFV genomes did not display the APOBEC-3G-specific mutations.

Controls performed in parallel with APOBEC-3G-negative 293T cells confirmed that Bet has no effect on the fidelity of the FFV provirus DNA synthesis.

### (B) Experimental System B and Results

A truncated form of HFV Bet extending only to Bet residue 279 and deleting all sequences to the end of Bet after amino acid 482 was constructed by filling in the Bg1II restriction site in bet resulting in an out-of-frame shift mutation in bet. In this regard reference is made to the wild type sequence of the HFV bet gene as published by Mariani et al., J. Virol. 1991, 65(2), pp. 727-735. The resulting plasmids “human and AGM (African green monkey) APOBEC3G-HA expression vector” (Mariani et al., Cell 2003, 114(1), pp. 21-31; available from The Salk Institute, La Jolla, USA) were transfected into BHK-21 cells. Again, virus particles were purified, DNA was extracted, a segment was amplified by PCR (sense-Primer: 5″-CTGCAGGATTGGATCCCCACAC-3″ (SEQ ID NO: 9); antisense-Primer: 5″-GCATATTGCAAAGCTGCATCACC-3″(SEQ ID NO: 10)), cloned and subsequently sequenced. Cotransfection of bet-inactivated HFV genomes together with APOBEC-3G resulted in APOBEC-3G-specific mutations (C to T on the minus provirus DNA strand) which were absent in the control when no APOBEC-3G was co-transfected. In addition, HFV genomes with intact bet genes displayed almost no APOBEC-3G-specific exchanges when human or simian APOBEC-3G was co-expressed.

### EXAMPLE 2

### Strong and Specific Interaction of HFV Bet and Human APOBEC-3G

Eukaryotic 293T cells were transfected with combinations of expression plasmids for human APOBEC-3G (containing an HA-tag for immuno-detection) [called: human APOBEC3G-HA expression vector, available from The Salk Institute, La Jolla, USA; Mariani et al., Cell 2003, 114(19; pp. 21-31) and HFV-Bet. HFV Bet was expressed from a CMV-promoter directly upstream of the spliced HFV bet coding sequence. The combinations were as follows: (a) no expression plasmids; (b) HFV-Bet only; (c) human APOBEC-3G only; and (d) human APOBEC-3G and HFV Bet.

Two days after transfection, cytoplasmic extracts were harvested and subjected to standard co-precipitation assays: Human APOBEC-3G was precipitated with an anti-HA antiserum (monoclonal antibody, HA11, CAT-Nr. MMS-101R, Berkeley Antibody Company, Richmond, Calif., USA) and the precipitated proteins were subjected to immuno-blotting using an anti-HFV Bet antiserum (Löchelt et al., Virology 184 (1991), pp. 43-54). HFV Bet was specifically precipitated by the anti-HA serum only in extracts of cells transfected with HFV Bet and APOBEC-3G. Without APOBEC-G3 or when using a heterologous antiserum, no Bet was precipitated. The HFV-Bet-APOBEC-3G complexes were even stable in 0.6 M NaCl indicative for a very stable protein-protein interaction.

### EXAMPLE 3

### Materials and Methods

Cell Culture and cDNA Preparation

FFV-permissive feline CRFKcells, FeFABcells, 293T cells, and FFV virions were propagated and used as described. Feline peripheral blood mononuclear cells (PBMCs) were isolated from EDTA-treated whole blood by Histopaque-1077 (Sigma) gradient centrifugation and cultured after activation with PHA (3 μg/ml) for 3 days in RPMI medium 1640 containing 15% FBS, 5×10⁻⁵ M 2-mercaptoethanol, 2 mM L-glutamine, and 100 units of human recombinant IL-2 per ml at 37° C. and 5% CO2. For cDNA preparation, total RNA was isolated by using the Rneasy minikit (Qiagen) according to the manufacturer. Total RNA (5 μg) was used to generate cDNA by using SuperScriptIII reverse transcriptase (Invitrogen).

### Plasmids and DNA Transfection

FFV WT and Bet mutant plasmids pFeFV-BBtr and pFeFV-MCS and the eukaryotic FFVBet expression plasmid have been described. In pFeFV-BBtr, the 387 residue WT Bet is truncated after amino acid 116, whereas in pFeFV-MCS, few residues are exchanged and inserted at the same site. To increase gene expression, both Bet mutations were cloned into the CMV-IE promoter-driven FFVpCF-7, resulting FFVpCF-7, resulting in mutant spCF-BBtr and pCF-MSC. The expression vector for hemagglutinin (HA)-tagged hu3G (phu3G-HA) was a gift of Nathaniel R. Landau (The Salk Institute for Biological Studies, LaJolla, Calif.). Feline APOBEC3 (fe3) was identified by using 5′ and 3′ RACE reactions (5′/3′-RACE kit, Roche Diagnostics) employing mRNA from CRFK cells, the forward fAPO3F9 (5′-TGGAGGCAGCCTGGGAGGTG-3′ (SEQ ID NO: 1)) and reverse fAPO3F16 (5′-CTTGAGGGAGGAGGGAGGATG-3′ (SEQ ID NO: 2)) primers, and Pwo polymerase (Roche Diagnostics). Thirty cycles were run at 94° C. for 30 s, 58° C. for 1 min, and 1 min, and 72° C. for 2 min. PCR products were cloned into pCR4 Blunt TOPO (Invitrogen), sequenced, and transferred into the EcoRI sites of pcDNA3.1(+) (Invitrogen) generating pfe3. Similarly, expression plasmid pfe3-HA encoding C-terminal HA-tagged fe3 was made by using forward fAPO3F18 (5′-TAGAAGCTTACCAAGGCTGGCGAGAGGAATGG-3′ (SEQ ID NO: 3)) and reverse fAPO3F19 (5′-AGCTCGAGTCAAGCGTAATCTGGAACATCGTATGGATACCTAAGGATTTCTTGAAGCTCTGC-3′ (SEQ ID NO: 4)) primers, sequenced, and cloned into the HindIII and XhoI sites of pcDNA3.1(+). DNA transfection into CRFK cells was done with Lipofectamine 2000 according to the manufacturer (Invitrogen), 293 T cells were transfected by Ca-phosphate precipitation.

The fe3 cDNA PCR product was inserted into the BamHI and SalI sites of bacterial expression plasmid pGEX4T3, and the glutationeS-transferase-tagged fe3 fusion protein was purified by glutathione Sepharose chromatography as described and used for antibody induction in rabbits.

### Virological Methods

FFV particles were prepared from infected CRFK cells 3 or 5 days after infection. Particles were enriched from cell culture supernatant by sedimentation through 20% sucrose and resuspended in PBS as described. Particles were digested with the subtilisin protease to remove proteinaceous contaminants not incorporated into the virions.

### Preparation of Particle-Derived Proviral DNA

To remove contaminating plasmid DNA, enriched FFV particles were treated for 2 h at 37° C. with DnaseI according to the supplier (MBI Fermentas, St. Leon-Rot, Germany). The Dnase was subsequently inactivated by adding EDTA to 2.5 mM, Proteinase K (Roche Diagnostics) to 0.2 mg/ml and incubation for 45 min at 72° C. Proteinase K was inactivated for 10 min at 98° C.

### PCR Amplification, Cloning, and Analysis of Proviral FFVDNA.

Virion-incorporated. FFV DNA was amplified with sense primer 5′-CTTCTGGTTTGGACCTTACC-3′ (SEQ ID NO: 7) and antisense primer 5′-GTTTTAGTAAGTGTAGCGGCGA-3′ (SEQ ID NO: 8) using the proof reading Herculase DNA polymerase according to the manufacturer (Amersham Pharmacia). A total of 34 reaction cycles were run at 94° C. for 30 s, 56° C. for 40 s, and 75° C. for 2 min. This PCR allowed amplification of unspliced FFV proviral DNA of ˜615 nt and spliced FFV proviral DNA of ˜330 nt and identification of the bet mutations. Reaction products were cloned by using the TOPO cloning kit as per the manufacturer's instructions (Invitrogen). Clones were identified by restriction enzyme digestion, and plasmid DNA was sequenced by using the DNA sequencer 377 (Applied Biosystems).

### Immunoprecipitation and Western Blot Analysis

For co-immunoprecipitation of FFV-Bet and fe3 or hu3G, 293 T cells were transfected with 2 μg off e3-HA or human APOBEC3G-HA expression plasmid pfe3-HA or phu3G-HA and 2 μg of pFFV-Bet. After 2 days, cells were lysed in TLB (20 mM Tris, pH7.4/137 mM NaCl/10% glycerol/2 mM EDTA, pH8/1% TritonX-100/50 mM Na-beta-glycerophosphate and protease inhibitors) and lysates cleared by centrifugation. For immunoprecipitation of fe3-HAorhu3G-HA, supernatants were incubated with anti-HA-beads (Roche Diagnostics) for 60 min at 4° C. and washed five times with TLB. After boiling in electrophoresis sample buffer, samples were subjected to SDS/PAGE and immunoblotting. The FFVBet, Env leader protein and cat 8014 antisera have been described. Membranes were reacted with horseradish peroxidase-conjugated secondary antibodies (Amersham Pharmacia) and visualized by enhanced chemiluminescence (ECL, Amersham Pharmacia). For immunoblotting, identical amounts of cell extracts were used as determined by Roti-Quant protein quantification (Roth, Karlsruhe, Germany).

### EXAMPLE 4

### Bet-Mutated FFV Genomes are Edited in Feline CRFK Cells

The inventors recently reported that CRFK cells display a nonpermissive phenotype when infected by bet-defective FFV. Similarly, Vif-minus feline immunodeficiencyvirus (FIV) is replication deficient in CRFK cells. In light of recent findings on the function of lentivirus Vif, it has been questioned whether expression of an APOBEC3-like cytidine deaminase in CRFK cells might be involved in the restriction of bet-deficient FFV.

Therefore, it has been re-examined the replication of the previously described FFVbet mutant spFeFV-MCS and pFeFV-BBtr in CRFK cells. In clone pFeFV-MCS, only a few amino acids in the central part of Bet had been changed, and clone pFeFV-BBtr is characterized by a truncation of Bet at the same site. As described, the changes in bet resulted in a 102- to 103-fold reduced titer of the mutants compared to WTFFV. To identify the cause for the reduced titer, de novo synthesized FFV genomes were analysed for the presence of APOBEC3-mediated C→U deamination of the DNA minus-strand resulting in G→A exchanges on the plus strand. For these studies, we took advantage of two specific features of FV reverse transcription: a substantial fraction of FV particles already contains full-length proviral DNA and part of this DNA specifically lacks the bet intron. These intron-deficient, bet-spliced FFVDNAs are only generated after replication of the plasmid-encoded FFVgenomes, and therefore cannot be derived from input DNA. CRFK cells were transfected with WT and bet-mutated FFV genomes. Released particles were purified 3 days later by sedimentation through sucrose and subjected to DnaseI digestion to remove plasmid DNA. The encapsidated, protected DNA was extracted and amplified by using PCR primers that allowed direct amplification of spliced and un-spliced FFV DNA and confirmation of the introduced bet mutations. FFVWT genomes displayed allow mutation frequency with no preference for G→A exchanges. In contrast, G→A substitutions were highly enriched in DNAs from both bet mutants, independent of whether spliced or unspliced DNA was sequenced. The number of G→A exchanges varied between 1 and 11 per sequence. Whereas all spliced cDNAs from both mutants contained at least one G→A exchange, some unspliced and thus even longer cDNAs of mutants pFeFV-MCS and pFeFV-BBtr did not. It is likely that these unmodified, full-length sequences were derived from input plasmid DNA and not from reverse-transcribed genomes despite the DnaseI digestion.

When analysing the minus strand for the sequence context in which the changes occurred, 68% were TTC to TTT changes, 14% were TCC to TCT exchanges, and in the remaining clones, at least one pyrimidine residue (NPyCorPyNC) preceded the altered C nucleotide. PyPyC to PyPyT mutations are typical for APOBEC3-mediated editing of retroviral genomes. In summary, these data indicate that CRFK cells express an APOBEC3-like deaminase (see below) and that FFVBet counteracts this editing activity.

To exclude the possibility that mutagenesis of bet interferes with the fidelity of FFV reverse transcription, we transfected WTpFeFV-7 and mutant pFeFV-BBtr into APOBEC-negative 293 T cells and analysed reverse-transcribed genomes from released particles for mutations. Under these conditions, the frequency and types of mutations were similar for WT and bet-mutated FFV genomes excluding a direct effect of Bet on the fidelity of the FFVRT (see below).

### EXAMPLE 5

### Characterization of Feline APOBEC3

To identify APOBEC3 expression in CRFK cells, degenerate primers derived from exons 3 and 6 of hu3G were used to amplify and clone the central part of the corresponding CRFK cell-derived feline APOBEC3 cDNA. The full-length feline APOBEC3(fe3) cDNA was subsequently constructed by 5′- and 3′-RACE techniques. The 192-aa-long fe3 shows significant homology to the second (48.5%) and first (38.1%) domain of hu3F and to the single domain of hu3C (46.4%) cytidine deaminases. hu3F and fe3 consistently have a similar editing preference for the trinucleotide TTC, whereas hu3G prefers CCC. When diagnostic PCR primers were used, substantial fe3 expression was detectable in CRFK cells and in PHA-activated feline PBMCs; the fe3 cDNA derived from PBMC was identical to that from CRFK cells.

### EXAMPLE 6

### Fe3 Reduces the Titer of bet-Deficient FFV and Induces Genome Editing

The effect of fe3 coexpression with WT and bet-deficient FFV genomes was studied after transfection of 293 T cells. For this purpose, the FFV titers were determined 2 days after transfection by using FeFAB reporter cells. Cotransfection of pfe3 reduced the WTFFV titer of pFeFV-7 up to 10-fold, whereas a102- to 103-fold reduction in titer was detected with the Bet-truncated pFeFV-BBtr mutant. This finding clearly demonstrates that FFVBet efficiently counteracts the antiviral activity of feline APOBEC3.

As described for CRFK cell-mediated FFV genome editing, a total of 29 FFV DNA genomes released from WT and bet-mutant pFeFV-BBtr cotransfected with either pfe3-HA or pUC18 was analyzed. Fe3 overexpression in 293 T cells resulted in 0.05 G→A exchanges per 100 nucleotides for the WTFFV genome compared with 0.13 G→A exchanges per 100 nucleotides when pUC control DNA was coexpressed. As expected, editing of the Bet mutant pFeFV-BBtr increased editing to 1.0 5 G→A exchanges per 100 nucleotides when fe3 was coexpressed, whereas few G→A exchanges (0.08 G→A exchanges per 100 nucleotides) occurred without fe3. The sequence context of the G→A exchanges by fe3 coexpression in 293T cells is similar to that seen in CRFK cells expressing the endogenous fe3 deaminase activity. This finding indicates that the majority of FFV genome editing in CRFK cells can be attributed to the cloned fe3 or a closely related feline cytidine deaminase.

### EXAMPLE 7

### FFVBet Specifically Binds to Feline APOBEC3

Because the fe3-encoded deaminase showed a Bet-dependent phenotype on FFV titer and genome editing, we analysed by co-immunoprecipitation assays whether fe3 is specifically bound by FFVBet. An FFVBet expression plasmid was co-transfected into 293 T together with plasmid pfe3-HA or control DNA cells, and lysates were subjected to co-immunoprecipitation using anti-HA beads, allowing detection of the HA-tagged fe3 protein. Similar to HIV-1 Vif, FFVBet was co-precipitated by fe3-HA, whereas FFVBet was not detected when the HA-tagged human APOBEC3G (hu3G-HA) protein was used, although it was clearly present in the lysate. These data demonstrate a species-specific binding of FFVBet to the homologous fe3 but not the heterologous hu3G protein.

### EXAMPLE 8

### FFVBet is not Incorporated in Virions

To determine whether the abundantly expressed cytoplasmic Bet that efficiently interacts with host cell-encoded fe3 is a component of viral particles, immunoblotting studies were performed. Particles were harvested from the supernatant of CRFK cells 5 days after WTFFV infection. The virions were subjected to subtilisin digestion to remove any Bet that was merely attached to the surface but not incorporated into FFV particles. Whereas low amounts of Bet were detectable in undigested FFV particles, subtilisin treatment completely eliminated Bet-specific signals, indicating that Bet was only copurified with virus particles. The conditions of subtilisin treatment were controlled by following digestion of the 16-kDa ectodomain of the FFVEnv leader protein (Elp) to the 9-kDamembrane-protected product. In lentiviruses, the APOBEC3-protecting Vif protein is found in released virions in most laboratories, but other groups have failed to detect Vif in virions.

### EXAMPLE 9

### Fe3 Interferes with FFV Particle Release and Accumulates in Particles from bet-Deficient Genomes

We analyzed whether fe3 expression affects FFV gene expression and release or composition of particle. To this end, WTpCF-7 and Bet mutant pCF-BBtr proviruses were cotransfected with decreasing amounts of plasmid pfe3-HA into 293 T cells. In cellular extracts, HA-tagged fe3 was clearly detectable as a discrete band of ˜22 kDa. The overall expression level of fe3-HA was not altered in WT versus mutant Bet-expressing cells. The expression level of FFV Gag was also not affected by fe3-HA co-expression; however, the processing of the FFV p52 Gag precursor to the p48 Gag cleavage product was consistently reduced on overexpression of fe3-HA, whereas Pol processing appeared normal. The observations that fe3 stability is not affected by Bet and that increasing amounts of fe3 interfere with Gag but not with Pol processing were confirmed in independent experiments.

We then analyzed the cell culture supernatants for WT and mutant particle release. When the Gag-reactive cat serum 8014 was used, co-transfection of high amounts (4 μg) of pfe3-HA strongly reduced release of particles derived from WT and bet-deficient proviruses, whereas lower amounts of fe3 only affected release from bet-deficient FFV genomes. A parallel blot reacted with the fe3-specific serum clearly revealed low amount of fe3-HA in particles from bet-deficient FFV genomes. In WT particles, miniscule amounts of fe3-HA were detectable only after overexposure of the blot. For pCF-BBtr-derived virus, the amount of fe3-HA detected paralleled the release of particles: the low-level release with high fe3 concentrations resulted in only trace amounts of fe3 in the particle fraction, whereas moderate particle budding (at 1 μg of pfe3-HA DNA) was paralleled by an increased fe3 release. These data show that WT Bet inhibits fe3 packaging into FFV particles.

### EXAMPLE 10

### Foamy Virus Bet Protein-Mediated Protection of Retroviral Vectors Against APOBEC3 Genome Editing

As a proof of principle, the effect of the human foamy virus (HFV) Bet protein was assayed for its capacity to protect retroviral vectors against APOBEC3-mediated inactivation.

In transient co-transfection assays, the HFV Bet efficiently protected human and simian immunodeficiency virus-derived retroviral vectors against functional inactivation by different primate APOBEC3 proteins (e.g. from chimpanzee, African green monkey, human). The read-out of these assays was the Bet-mediated rescue of marker gene transduction in the presence of different APOBEC3 molecules. The Bet-mediated rescue was up to 100-fold and depended on the vectors and APOBEC3 proteins used.

### Summary of Results:

The data presented indicate that FFV Bet binds to fe3. Together with the high-level cytoplasmic expression of Bet in all cell culture systems studied, this points to an active sequestration of APOBEC3 away from the sites of FV particle assembly. This active sequestration of fe3 is in line with the observation that functional inactivation of Bet correlated with accumulation of fe3 in released virus particles. A possible alternative mechanism is that Bet directs APOBEC3 proteins to proteasome-mediated degradation as is well documented for Vif. The fact that subtle mutations of Bet in clone pFeFV-MCS destroyed its protective potential as severely as truncating Bet at the same site indicates that this central part of Bet either directly affects its function, e.g., during APOBEC3 binding, or that this sequence is absolutely required for proper protein folding. The high concentrations of Bet may be not only required for APOBEC3 sequestration, but also to the other Bet functions, e.g., in establishing and maintaining persistence, reactivation from latency, intercellular trafficking, or particle release.

The most distinguishing feature in the APOBEC3G-mediated editing of FV genomes in contrast to orthoretroviruses is the timing of deamination: in orthoretroviruses, editing only occurs in the newly infected cell. In contrast, deamination of FFV genomes by fe3 is already clearly detectable in genomes packaged into released particles. We obtained similar data for the primate FV that can be edited by different APOBEC3 deaminases. The early onset of FV genome editing is most probably related to the fact that FV reverse transcription already starts before or during particle formation and release in the virus-producing cell. This finding may explain our observation that only low amounts of fe3 are present in particles from bet-deficient FFV genomes, because, in FVs, the virus-producing cell, and not the newly infected cell, is the major site of APOBEC3 action.

What is claimed is:

1. An in-vitro method of preventing the negative effects of an APOBEC3 enzyme for increasing the production and/or genetic stability of a viral vector in a cell, comprising administering a nucleic acid sequence encoding a FV Bet protein into the cell.

2. The method of claim 1, wherein the APOBEC3 enzyme is an APOBEC3G or APOBEC3F enzyme.

3. The method of claim 1, wherein the viral vector is a HIV vector or a foamy virus (FV) vector.

4. The method of claim 1, wherein the APOBEC3 enzyme is a primate APOBEC3 enzyme and the FV Bet protein is a human FV Bet protein.

5. The method of claim 4, wherein the primate APOBEC3 enzyme is a human or simian APOBEC3G enzyme.

6. The method of claim 1, wherein the APOBEC3 enzyme is a primate APOBEC3 enzyme, the FV Bet protein is a human FV Bet protein and the viral vector is selected from the group consisting of human or simian immunodeficiency virus-derived vectors and human FV vectors.

7. The method of claim 1, wherein the APOBEC3 enzyme is a feline APOBEC3 enzyme and the FV Bet protein is a feline FV Bet protein.

8. The method of claim 1, wherein the APOBEC3 enzyme is a feline APOBEC3 enzyme, the FV Bet protein is a feline FV Bet protein and the viral vector is a feline FV vector.


SEQUENCE LISTING (10 sequences)

SEQ ID NO: 1 - DNA, 20 bp, artificial
  forward fAPO3F9
tggaggcagcctgggaggtg

SEQ ID NO: 2 - DNA, 21 bp, artificial
  reverse fAPO3F16
cttgagggaggagggaggatg

SEQ ID NO: 3 - DNA, 32 bp, artificial
  forward fAPO3F18
tagaagcttaccaaggctggcgagaggaatgg

SEQ ID NO: 4 - DNA, 62 bp, artificial
  reverse fAPO3F19
agctcgagtcaagcgtaatctggaacatcgtatggatacctaaggatttcttgaagctctgc

SEQ ID NO: 5 - DNA, 20 bp, artificial
  sense primer
cttctggtttggaccttacc

SEQ ID NO: 6 - DNA, 22 bp, artificial
  antisense primer
gttttagtaagtgtagcggcga

SEQ ID NO: 7 - DNA, 20 bp, artificial
  sense primer
cttctggtttggaccttacc

SEQ ID NO: 8 - DNA, 22 bp, artificial
  antisense primer
gttttagtaagtgtagcggcga

SEQ ID NO: 9 - DNA, 22 bp, artificial
  sense primer
ctgcaggattggatccccacac

SEQ ID NO: 10 - DNA, 23 bp, artificial
  antisense primer
gcatattgcaaagctgcatcacc